(19) United States
(12) Patent Application Publication
(10) Pub. No.: US 2010/0104641 A1
(43) Pub. Date: Apr. 29, 2010

(54) THERAPEUTIC COMPOSITION, AND USE OF A CELL-FREE SUBSTANCE

(75) Inventors: Michael Thie, Bestwig (DE); Oezer Degistirici, Bonn (DE)

Correspondence Address: JOYCE VON NATZMER;PEQUIGNOT + MYERS LLC, 200 Madison Avenue, Suite 1901, New York, NY, 10016, US

(73) Assignee: STIFTUNG CAESAR, Bonn (DE)

(21) Appl. No.: 12/525,481

(22) PCT Filed: Feb. 1, 2008
(86) PCT No.: PCT/DE2008/000184

§ 371 Date: Dec. 16, 2009

(30) Foreign Application Priority Data
Feb. 1, 2007 (DE) 10 2007 005 946.0

(51) Int. Cl. A61K 35/32; A61K 35/12; A61K 33/42

(52) U.S. Cl. 424/484; 424/520; 424/549; 424/602

(57) ABSTRACT

The invention relates to a therapeutic composition, a method for producing a therapeutic composition, and the use of a cell-free substance, especially a cell-free bone or cartilage matrix. The disclosed therapeutic composition comprises at least a cell-free substance obtained from stimulated stem cells and/or precursor cells. Immunogenic reactions during in vivo therapeutic use are prevented by the fact that the therapeutic composition is free from cells and contains no typically antigenic cell components. The disclosed composition can therefore be universally used for the therapeutic purposes regardless of the origin of the stem cells and/or precursor cells and utilize the natural regenerative potency thereof in a highly efficient manner for replacing tissue, e.g. for a suitable bone and/or cartilage structure.

### CROSS-REFERENCE TO RELATED APPLICATIONS

This is the U.S. national stage of International application PCT/DE2008/000184, filed Feb. 1, 2008 designating the United States and claiming priority from German application DE 10 2007 005 946.0, filed Feb. 1, 2007.

### FIELD OF THE INVENTION

The invention relates to a therapeutic composition, a method for producing a therapeutic composition as well as the use of a cell-free substance, especially a cell-free bone or cartilage matrix.

### BACKGROUND OF THE INVENTION

The reconstitution of larger osseous defects and smaller defects in oral surgery/orthopedics still is a serious therapeutic problem. In the past, the insertion of artificially produced replacement materials (bio-materials) into the osseous defect zone only resulted in unsatisfying results. Thus, larger osseous areas filled with hydroxylapatite (HA) are biochemically instable and involve the risk of fracture. Further undesired effects of implanted bio-materials are a chronically aseptic inflammatory reaction and an increased local risk of infection caused by a disturbance of the body's own immunity condition (Vacanti et al., 1998).

Due to a lack of alternatives (like e.g. the sole use of bio-materials with a low biological value), autologous bone and bone marrow transplants (cancellous bone) still are the standard (Gerngross et al., 1982; Jerosch & Plewka, 1992; Rueger, 1998). This method, however, requires a second surgery, and in approx. 55% of all surgeries results in permanent discomfort. The considerably longer healing periods of this therapy result in an additional burden for the health care system. Furthermore, autologous bone substance is available very limitedly only (Wippermann et al., 1997).

Stem cells are cells of the body, which are not differentiated, i.e. stem cells have not yet been specialized for a task in the organism, for example as a skin or liver cell. From stem cells, further stem cells and/or differentiated cells can be obtained by division, i.e. stem cells are able to divide asymmetrically. In that, a stem cell retains its ability to divide over a very long period, often even for the entire life of the organism. Triggered by specific signals during the development of the organism, a stem cell can differentiate into different cell types, which then form the organism. There is a general differentiation between embryonic and adult stem cells.

Embryonic stem cells (ES cells), which are obtained from an embryo up to the 8-cell stage, are called totipotent. From these cells, the entire organism (human being) develops. Embryonic stem cells from later blastocyst stages are called pluripotent, since from them normally any types of body cells of the endoderm (wall cells of the digestive tract), mesoderm (muscles, bones, blood cells) and ectoderm (skin cells and nerve tissue) can still differentiate, but not a complete organism anymore. For ethical reasons and due to problems with the molecular control of cell differentiation, however, ES cells are not therapeutically applicable so far.

Adult stem cells (AS cells), on the other hand, are generated after the embryonic stage, i.e. are undifferentiated cells, which are located in a differentiated tissue and from which specialized cells originate, which correspond those of the differentiated tissue. ES cells, however, can also differentiate into cell types, which are to be allocated to another tissue. Adult stem cells, which can be found in organs, in the bone marrow or also in the umbilical cord, however, cannot differentiate as freely as embryonic stem cells anymore. Although adult stem cells do not have the same differentiation potential like embryonic stem cells, they still have a differentiation potential exceeding the germ layer. Therefore they are called multipotent. Thus, for example mesenchymal stem cells, originating from the mesoderm, can also differentiate into neuronal cells, which otherwise develop from the ectoderm. After relocation into another tissue type, AS cells are thus able to differentiate into a cell type, which does not correspond to that of their stem tissue. Adult stem cells are available in every organ, for example in the bone marrow. The withdrawal of bone marrow, however, is a complicated and risky operating technique. Contrary to that, the obtainment of stem cells from dental tissue is a less intricate alternative, like described for AS cells from dental follicle in WO 03/066840 A2. Such stem cells from easily accessible tissue open up, for example, the perspective of tissue replacement with the body's own cells. Also, the tendency for malign degeneration seems to be less upon implantation of adult stem cells than with embryonic stem cells. Adult stem cells are thus of increasing significance for the development of innovative therapeutic approaches.

From EP 0 957 944 B1, a method for producing a bone matrix colonized with osteogenic cells is known, which after implantation in vivo is to stimulate the bone regeneration by induction of differentiation processes in the cells of the surrounding tissue. For this purpose, first, osteogenic precursor cells are isolated from explants of somatic cells of the skin, the cartilage, the bone or the dental system, in particular human stem cells from the iliac crest. The isolated cells are stimulated in vitro by adding growth factors, for example bone morphogenetic proteins (BMPs), and thus the synthesis of bone matrix is initiated. The bone matrix may then be separated from the cells by enzymatic treatment or freezing and subsequent washing, so that a cell-free bone matrix results. This cell-free bone matrix is then re-colonized with fresh osteogenic cells, whereby an autologous, cell-loaded bone replacement is generated. Thus, with this method, osteogenic cells and bone matrix are produced separately and subsequently reunited for producing a bone replacement material. The use of a cell-containing bone replacement, however, has the disadvantage, that due to the antigenic properties of the living cells, this may result in immunogenic rejection reactions, if the cells were not obtained from the person to be treated him-herself. Such a cell-loaded bone matrix is consequently not universally applicable in therapy.

### SUMMARY OF THE INVENTION

It is therefore an object of the invention to create a therapeutic composition for highly efficient preparation of patients, who due to deficits in the tissue structure cannot, or only with difficulties, be provided with implants.

According to the invention, the object is solved by a therapeutic composition, which comprises at least a cell-free substance obtained from stimulated stem and/or precursor cells. Immunogenic reactions during in vivo therapeutic use are prevented by the fact that the therapeutic composition is free from cells and contains no typically antigenic cell components. The composition according to the invention can therefore be universally used for therapeutic purposes regardless of the origin of the stem cells and/or precursor cells, and utilize the natural regenerative potency thereof in a highly efficient manner for replacing tissue, e.g. for a suitable bone and/or cartilage structure. The therapeutic composition according to the invention can stimulate the regeneration of destroyed tissue in vitro and in vivo, although it does not contain any stem cells itself anymore. Obviously, the substances contained in the stimulated stem cells and/or precursor cells or produced by these cells are sufficient to enable an effective tissue replacement therapy or to at least support it. In that, the composition according to the invention does not comprise any antigenic cell components and may thus be applied in all patients without the risk of immunogenic reactions. This enables an industrial production of the preparation and thus ensures permanent availability.

In an advantageous embodiment of the invention, the cell-free substance is a cell culture, in which the living cells have been killed and/or cell components were at least partially removed. For that, any antigenic components, for example antigens of the cell surface or other proteins, must be effectively removed. Nucleic acids should likewise be removed or at least completely degraded in order to exclude possible risks by the insertion of genetic material.

In a particular embodiment of the invention, the cell-free substance comprises cell extract and/or cell secretion, i.e. antigen-free cell components, cell materials and/or substances produced by the stimulated cells.

In a particularly advantageous embodiment of the invention, the cell-free substance is developing bone matrix. The formation of this matrix takes place on the basis of ectomesenchymal precursor cells and in its nature is close to that of the embryonic bone formation, so that the composition according to the invention in respect of the growth-promoting modulators can be applied or used, respectively, in a bone replacement therapy (catalyst for the bone structure). In that, the cell-free bone matrix enables a targeted stimulation of the bone structure in vivo, without having to reckon with immunogenic reactions. Thus, for example patients, who due to deficits in the bone structure cannot, or only with difficulties, be provided with implants, e.g. dental implants, can be prepared in a simple manner and without any risk by insertion of the therapeutic composition into the defective tissue. In addition, or alternatively, the cell-free substance may also be a cartilage matrix, so that by means of the composition of the invention defective cartilage tissue may be regenerated as well. The therapeutic composition may also be used for the treatment of injuries or degenerative diseases in joints.

In an advantageous embodiment of the invention, the stem cells and/or precursor cells forming the cell-free substance can be multipotent cells from a soft tissue of a tooth. Preferably, stem cells from follicle or pulp may be used. Particularly preferred are stem cells from a pad-like soft tissue, which can be localized immediately at the apical side of an extracted immature tooth below the papilla. Advantageously, the pad-like soft tissue is a cell formation from a disposition of an impacted and/or retained tooth in the development phase between the occurrence of the osseous alveolar fundus and the completion of the root formation. In order to be able to isolate the desired multipotent stem cells, the pad-like soft tissue should, after surgical removal of the tooth, be separated from the tooth along a macroscopically visible border between the pad-like soft tissue and the papilla, preferably below an imaginary line between the developing rootlets. The tissue selected in such manner allows for the isolation of ectomesenchymal stem cells or precursor cells, respectively, which due to their multipotency can be differentiated into different cell types, for example bone or cartilage cells. The selection of the right tissue of origin for the isolation of the stem cells according to the method of the invention is a substantial factor for a high yield of multipotent stem cells. The isolated cells are ectomesenchymal stem cells and/or precursor cells, which can be stimulated osteogenically and/or chondrogenically after isolation from the tissue formation.

In a particularly advantageous embodiment of the invention, the cell-free substance has a defined ratio of calcium to phosphate. With the targeted setting of a defined calcium/phosphate ratio, for example bone matrix can be produced, which corresponds to a young bone or an old bone. Depending on how long the stem cells are cultivated under osteogenic stimulation, the cell-free substance according to the invention may thus be targetedly tailored to the desired application. The calcium/phosphate ratio provides references to the age of the bone (Dorozhkin, S. V., Epple, M. 2002). Consequently, according to the invention, a therapeutic composition can be provided, which comprises a cell-free substance with a defined calcium/phosphate ratio, preferably a calcium/phosphate ratio between 0.5 and 2.0, so that a remedy optimized for the desired therapy is present, which has defined properties. Furthermore, the cell-free substance may also have a defined ratio of calcium phosphate to collagen, so that the quality of the therapeutic composition can also be set exactly.

The object is furthermore solved according to the invention by a method for producing a therapeutic composition, in which stem cells and/or precursor cells are isolated, cultivated in vitro and stimulated for subsequent differentiation, wherein after differentiation, the cells are killed and/or at least partially removed for producing a cell-free substance, and whereat the cell-free substance is prepared for therapeutic use. Thus, according to the invention, contrary to the state of the art, a cell-free substance, which was obtained or produced, respectively, from stimulated stem cells and/or precursor cells, is directly brought into a therapeutic form of application without colonizing it with stem cells again. Surprisingly, it turned out that the therapeutic composition produced according to the method of the invention can stimulate the regeneration of destroyed tissue in vitro and in vivo, although it is not colonized with fresh stem cells. Obviously, the substances contained in the stimulated stem cells and/or precursor cells or produced by these cells are sufficient to allow for an effective tissue replacement therapy or to at least support it. The production method is thus considerably facilitated and additionally more cost-effective, since in parallel to the generation of the cell-free substance, no stem cells or precursor cells have to be cultivated anymore.

According to the invention, the cell-free substance is preferably disintegrated, pulverized and/or freeze-dried, so that a product results, which can be easily handled and processed. The cell-free substance may then, according to the invention, be prepared, for example in the form of a paste, an ointment or a suspension. In this or in a similar form, the cell-free substance or the therapeutic composition can be inserted into the tissue to be treated in a simple manner. This gentle type of application is of particular advantage, since through this, additional interventions burdening the patient can be avoided.

In a particularly advantageous embodiment of the method according to the invention, the stem cells and/or precursor cells are osteogenically and/or chondrogenically stimulated. In that, the stem cells and/or precursor cells are stimulated in such manner that they form or produce, respectively, a bone or cartilage matrix. This matrix and any other substance formed in vitro without additional auxiliary means, like for example substrates, may then be liberated from living cells and/or antigenic cell components by enzymatic, chemical or mechanical treatment and subsequently used for the production of the composition according to the invention. In that, living cells should at least be killed and/or antigenic cell components removed as completely as possible. Nucleic acids should likewise be removed or at least completely degraded in order to exclude possible risks by inserting genetic material. Preferably, the intact or disintegrated bone mass is treated with a hypotonic solution (e.g. de-ionized water) and/or treated in liquid nitrogen for ice crystal formation in order to destroy the cell membranes and to lyse the cells. Depending on the degree of maturity of the bone tissue and the composition of the later product desired therewith, the samples are washed under shaking in saline solution (e.g. 3M NaCl) and/or acid/base (e.g. 0.15-1% peracetic acid—PAA), and/or extracted in Triton-X100 (0.1%-1%) and/or SDS (0.1%-1%) for differently long periods. Intermediate steps for biochemical modification of the organic bone base substance with enzymes (e.g. trypsin/EDTA) are optionally possible. Intermediate steps for chemical modification of the inorganic base substance with acids/bases are likewise possible. In order to remove released nucleic acids (RNA and DNA), the bone base substance is treated with RNAse and DNAse. Subsequently, the cell-free bone base substance is washed with buffer solution (e.g. PBS), dialyzed and lyophilized, pulverized and prepared in the form of a paste, an ointment or a suspension.

According to the invention, the duration of the stimulation for setting certain properties of the cell-free substance can be varied. In a particularly advantageous embodiment of the method according to the invention, with the selection of a certain duration of stimulation for example, a defined ratio of calcium to phosphate and/or of calcium phosphate to collagen can be set. The duration of stimulation, i.e. the duration of cultivation of the stem cells in the presence of the stimulated substances, according to the invention, lies between 15 and 50 days, preferably between 20 and 45 days, particularly preferred between 21 and 42 days. With the targeted selection of a certain duration of stimulation, the degree of maturity and the quality of the cell-free substance can be influenced and thus set in a defined manner. The shorter the duration of stimulation, the lower the degree of maturity or mineralization, respectively (low calcium/phosphate ratio) of the produced bone matrix or cell-free substance, respectively. The longer the duration of stimulation, the higher the degree of maturity or mineralization, respectively (high calcium/phosphate ratio) of the produced bone matrix or cell-free substance, respectively. The method according to the invention thus enables in a particularly advantageous manner the targeted setting of the properties of the process product with the selection of a certain duration of stimulation.

In a further particularly advantageous embodiment of the method according to the invention, the stem cells are isolated from a soft tissue of a tooth, like for example follicle or pulp. Preferably, the stem cells from a pad-like soft tissue are isolated, which can be localized immediately at the apical side of an extracted immature tooth below the papilla. In respect of the multipotency of the stem cells to be isolated within the course of the method according to the invention, it is particularly advantageous, if the pad-like soft tissue is obtained from a disposition of an impacted and/or retained tooth in the development phase between the occurrence of the osseous alveolar fundus and the completion of the root formation. In order to be able to isolate the desired multipotent stem cells, after surgical removal of the tooth, the pad-like soft tissue should be separated from the tooth along a macroscopically visible border between the pad-like soft tissue and the papilla, preferably below an imaginary line between the developing rootlets. The tissue selected in this manner allows for the isolation of ectomesenchymal stem cells or precursor cells, respectively, which due to their multipotency can be differentiated into different cell types, for example bone or cartilage cells. The selection of the right tissue of origin for the isolation of the stem cells according to the method of the invention is a substantial factor for a high yield of multipotent stem cells. The tissue formation can be dissolved by enzymatic treatment, preferably with collagenase/dispase, for further cultivation of the cells. In that, the cells may also be singularized after obtainment from the tissue formation. The cells isolated using the method according to the invention are ectomesenchymal stem cells and/or precursor cells, which can be osteogenically and/or chondrogenically stimulated in vitro after isolation from the tissue formation.

Any therapeutic composition produced according to the method of the invention is likewise subject of the invention. In particular any cell-free bone and/or cartilage matrix produced using the method described above.

In a particularly advantageous embodiment of the invention, the cell-free substance described above may be used, as explained already, for therapeutic purposes within the scope of tissue replacement therapy. The invention furthermore comprises the use of a cell-free cell extract and/or cell secretion for therapeutic purposes within the scope of tissue replacement therapy as well as the use of a cell-free bone or cartilage matrix for therapeutic purposes within the scope of tissue replacement therapy.

In the following, the invention will be explained in more detail on the basis of the figures and exemplary embodiments.

### BRIEF DESCRIPTION OF THE FIGURES

In the figures,

FIG. 1 shows (a) a photograph of an extracted wisdom tooth with apical soft tissue (apical cushion) and (b) a micrograph of cells isolated from the apical cushion,

FIG. 2 shows scanning electron micrographs of bone formations after 28 days of incubation of the isolated cells after glutaraldehyde fixation (coated with gold), (a) control, (b) exterior view and (c) cross-sectional view,

FIG. 3 shows micrographs of osteogenically stimulated cultures after immunohistochemical detection of (a) osteocalcin (company Acris), (b) collagen type I (company Abcam) and (c) after staining with alizarin red.

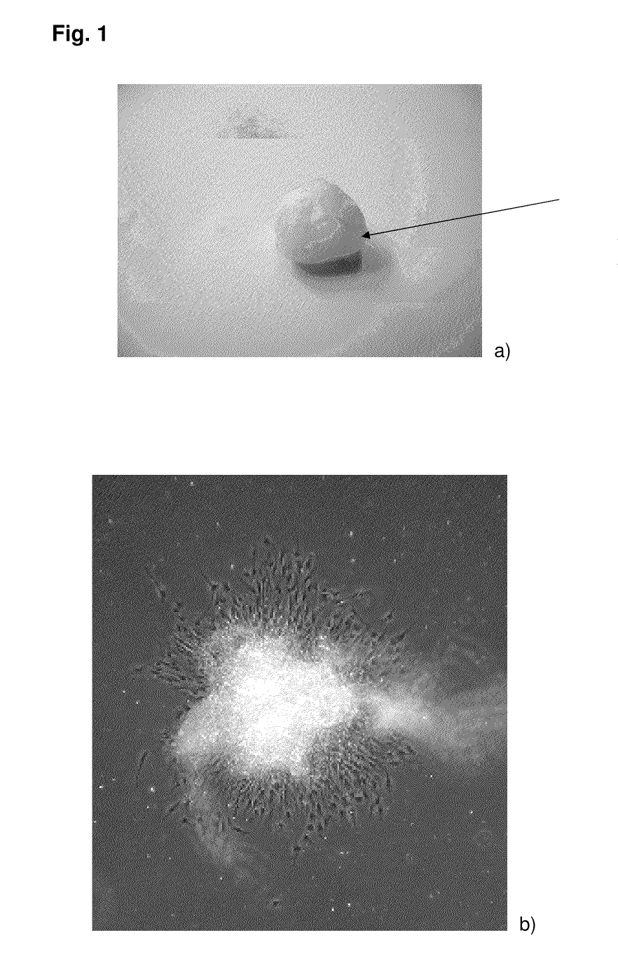

FIG. 1 shows a surgically removed wisdom tooth with an apical cushion (a). The apical cushion is separated from the tooth along the macroscopically visible border between the pad-like soft tissue and the papilla below the imaginary line between the roots of the tooth, washed several times with PBS (sterile) and then disintegrated with a scalpel. The tissue thus obtained is digested with collagenase/dispase. After the treatment of the apical soft tissue with collagenase/dispase, the cultures are cultivated at 37° C. in DMEM LG 10% FCS. The isolated cells grow as fibroblastoid cells in cultures (b).

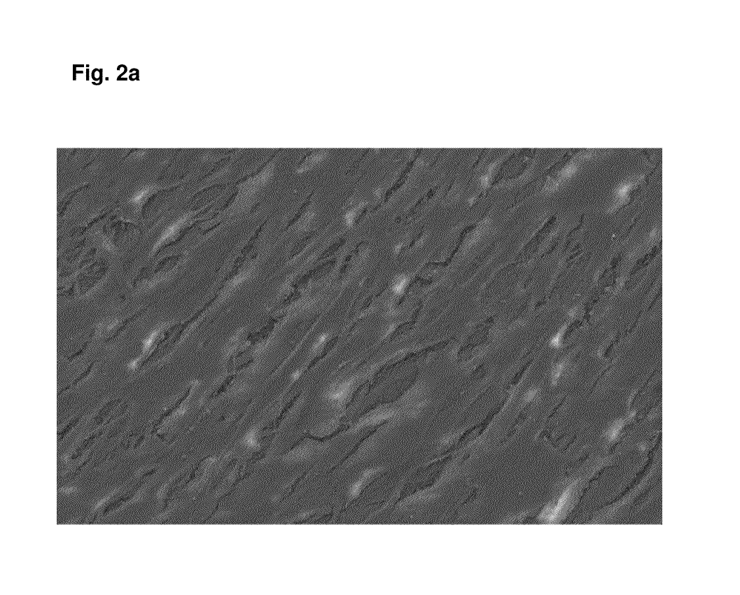

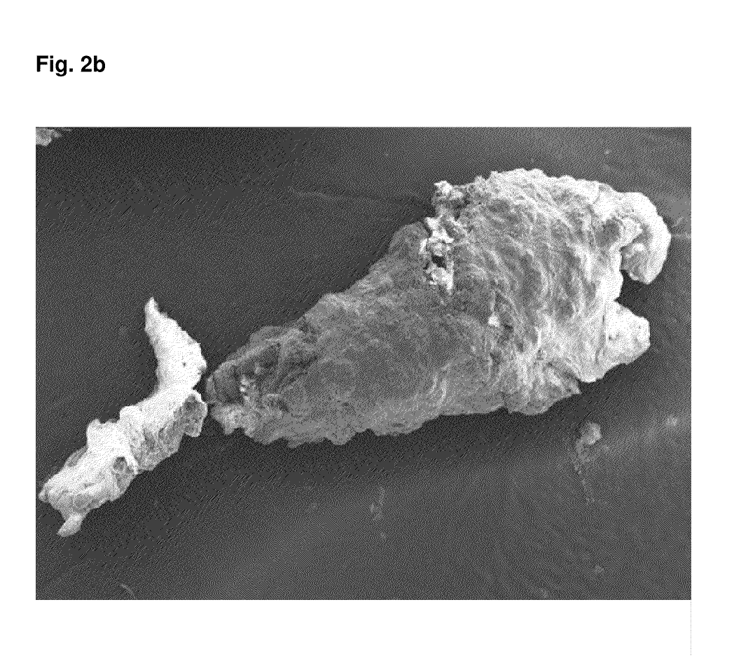

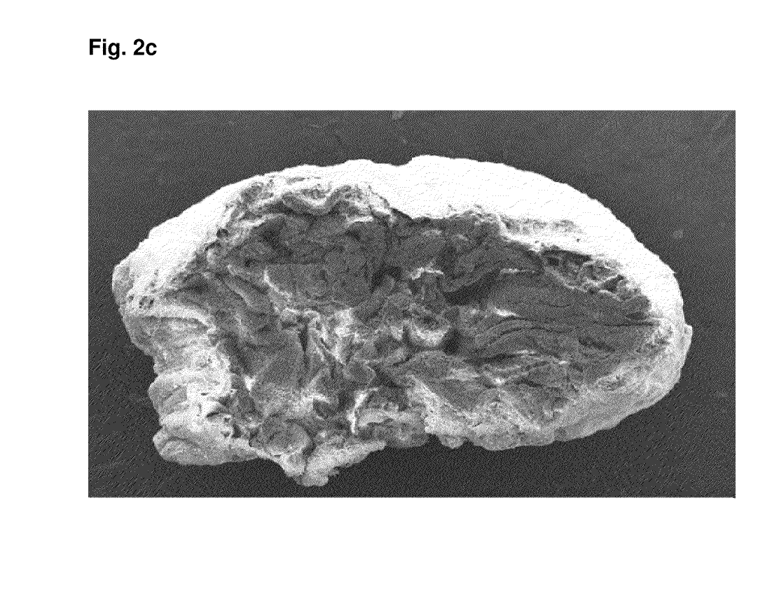

FIG. 2 shows scanning electron micrographs of bone formations after 28 days of incubation of the isolated cells. The cells are cultivated in control medium (a) and in osteogenically stimulated medium (b). The cells cultivated in osteogenically stimulated medium form a bone-like structure (b), which organized from the inside is structured bone-like (c), while the cells, which were only treated with control medium, form no bone structure and grow as an adherent formation (a).

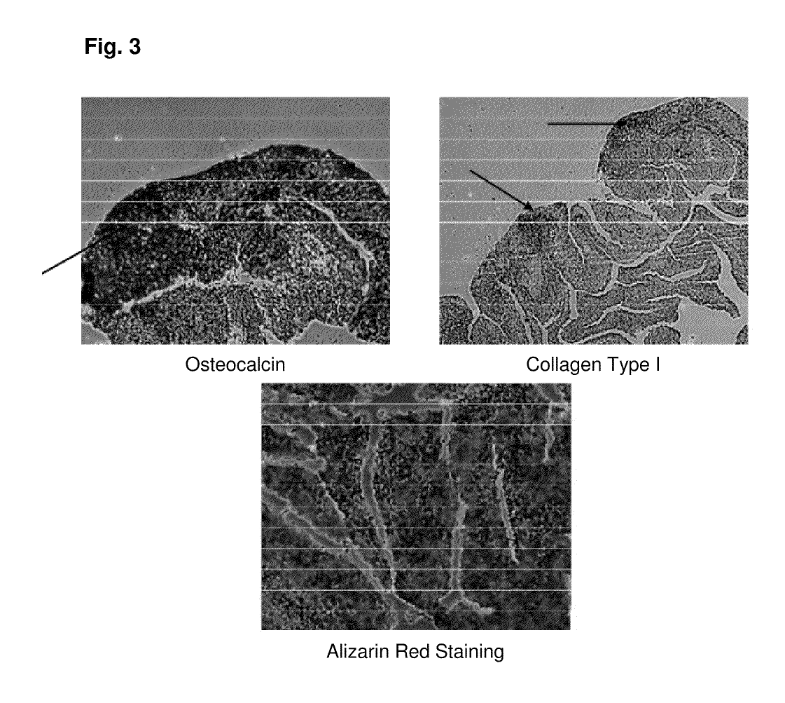

FIG. 3 shows micrographs of osteogenically stimulated cultures. The osteogenically stimulated cultures are stopped after 42 days and fixed with 4% PFA. Subsequently, the formed bone structure is embedded in paraffin and then cut. In order to detect the bone-like structures, the cuts are immunohistochemically stained with a first antibody against osteocalcin or collagen type I (both are bone markers). The bound antibodies are detected with the DAB Kit according to the manufacturer's instructions (company R&D). In order to detect calcification, the cuts are stained with alizarin red (1.2% alizarin red with pH 4.2). The areas with high red staining are calcified or bone-like areas, respectively.

### PREFERRED AND EXEMPLARY EMBODIMENTS OF THE INVENTION

Dental stem cells have a specific pattern of active substances which control the setup of various tissue types. In order to use this potency, an especially isolated subpopulation of human dental stem cells is stimulated in a cell-biological process to develop a tissue formation similar to that of a young bone. The thus formed highly specific matrix is subsequently prepared into a non-cellular product, which significantly accelerates the bone formation and thus the course of the healing phase and the in-growth behavior of implants. Thus, the special property of this matrix is used to stimulate, due to its composition, the complex bone formation in a wound in a physiologically correct manner.

In the first step, stem/progenitor cells are osteogenically stimulated under suitable conditions to make them form a bone-like tissue formation out of themselves. The osteogenic stimulation may, for example, take place by incubation or cultivation, respectively, of the stem cells with 10⁻⁷ M dexamethasone, 50 μg/ml ascorbic acid 2-phosphate and 10 mM β-glycerol-phosphate. This biological construct is in its composition comparable with the forming bone. Therefore, in the second step, the matrix is prepared and processed into a product. The entire process is purely biological and does without any substrate (scaffold).

As the starting material serves a differentiated bone tissue, as it can only be formed from young dental stem cells, the so-called dNC-PCs, which are osteogenically stimulated, in a self-generating culture system. The culture system is particularly advantageous, because the three-dimensional bone tissue, including its specifically formed intercellular substances, is exclusively organized by dNC-PCs themselves and in its structure and function is exclusively subject to its own forming and ordering principles. Thus, the bone tissue is an immature bone tissue to be, which in its structure and strength, its degree of mineralization as well as its composition of base substance components is of a special construction. This bone tissue is prepared into a non-cellular product (bone base substance), which accelerates the bone formation and thus the course of the healing phase and the in-growth of implants (scaffolds) in the patient. This bone tissue can be harvested in different degrees of maturity, which depend on the phases of the matrix formation and the mineralization, and prepared in processes depending on the degree of maturity. The degrees of maturity are determined on the basis of the differentiation conditions of the dNC-PCs or the structure and composition of the matrix, respectively.

The removal of the cells and the production of a cell-free substance take place with physical, chemical and biological methods. In order to destroy the cell membranes and to lyse the cells, the intact or disintegrated bone mass is treated with a hypotonic solution (e.g. de-ionized water) and/or treated in liquid nitrogen for ice crystal formation. Depending on the degree of maturity of the bone tissue and the thus desired composition of the later product, the samples are washed under shaking in saline solution (e.g. 3M NaCl) and/or acid/base (e.g. 0.15-1% peracetic acid—PAA), and/or extracted in Triton-X100 (0.1%-1%) and/or SDS (0.1%-1%) for differently long periods. Intermediate steps for biochemical modification of the organic bone base substance with enzymes (e.g. trypsin/EDTA) and/or chemical modification of the inorganic base substance with acids/bases are likewise possible. In order to remove released nucleic acids (RNA and DNA), the bone base substance is treated with RNAse and DNAse. Subsequently, the cell-free bone base substance is washed with buffer solution (e.g. PBS), dialyzed and lyophilized, pulverized and prepared in the form of a paste, an ointment or a suspension.

The cell-free substance is thus principally based on the synthesis performance of a neural crest cell. These cells are isolated from the cells of a pad-like soft tissue, which can be localized immediately at the apical side of an extracted immature tooth below the papilla (where they have sedimented in their niche) and described in their properties as multipotent dNC-PCs (=dental neural crest-derived progenitor cells). Only this particular cell type, i.e. the dNC-PCS, can form a three-dimensional, purely biological cell matrix aggregate under osteogenic stimulation. This aggregate, which forms itself completely independently, in general comprises a “core” and a “shell”. The core is formed by interior cells, the shell by exterior cells. Both cell types can be distinguished phenotypically, however, initially originate from dNC-PCs as a common precursor. The core of the aggregate comprises vital cells, which are all related and belong to the family of osteoblasts and osteoblast helper cells. The shell of the aggregate likewise comprises vital cells. The exterior cells have a barrier function to screen off the interior of the aggregate from the outside world (=medium, factors, serum) and to allow for the setting of a specific milieu. With increasing duration of the culture, the interior cells start to secrete an embryonic matrix, which first is not mineralized. Only with long culture periods, there is a gradual deposition of minerals (variants of Ca phosphates up to hydroxylapatite) and thus the formation of hard substance. The process of mineralization takes place in the interior of the aggregate only and is probably bound to the presence of the shell. Mineralization is an active process, i.e. it depends on the interior cells and the special milieu of the core. The interior of the aggregate is the initial product for the cell-free substance according to the invention, which can be harvested in different degrees of maturity. For harvesting, the aggregate is broken open. The exterior cells are removed via enzyme treatment or also immune-surgery. What is left is the core of the aggregate, which is not used directly, but is still prepared. The cells are isolated with enzyme and detergent. What is left can be separated into an organic and an inorganic phase. Each phase can be modified further—the organic one with enzyme, the inorganic one with acid and/or base. Depending on the degree of maturity of the aggregate and depending on the preparation process, this respectively results in a different product.

### LITERATURE

Gerngross H, Burri C, Kinzl L, Merk J, Muller G W.
Complications at removal sites of autologous cancellous bone transplants
Aktuelle Traumatol. 1982 June; 12(3):146-52.
Jerosch J, Plewka U.
Medical applications of electronic data processing in surgery, trauma surgery and orthopedics. Results of a survey of 1,450 clinics
Z Orthop Ihre Grenzgeb. 1992 September-October; 130(5):390-8.
Rueger K.
Diagnosis and therapy of malignant lymphedema
Fortschr Med. 1998 Apr. 30; 116(12): 28-30, 32, 34. German.
Vacanti J P, Langer R, Upton J, Marler J J.
Transplantation of cells in matrices for tissue regeneration.
Adv Drug Deliv Rev. 1998 Aug. 3; 33(1-2): 165-182.
Wippermann C F, Schmid F X, Kampmann C, Eberle B, Brandey I, Schranz D, Huth R G.
Evaluation of gastric intramucosal pH during and after pediatric cardiac surgery.
Eur J Cardiothorac Surg. 1997 August; 12(2):190-4.
Dorozhkin, S. V.; Epple, M. ANGEWANDTE CHEMIE-INTERNATIONAL EDITION 2002, 41, 3130-3146.

1. A therapeutic composition comprising at least a cell-free substance obtained from stimulated stem cells and/or precursor cells.

2. The therapeutic composition according to claim 1, wherein said cell-free substance is a cell culture, in which the living cells have been killed and/or cell components were at least partially removed.

3. The therapeutic composition according to claim 1, wherein said cell-free substance comprises cell extract and/or cell secretion.

4. The therapeutic composition according to claim 1, wherein said cell-free substance is bone or cartilage matrix.

5. The therapeutic composition according to claim 1, wherein said stem cells are multipotent cells from a soft tissue of a tooth, preferably follicle or pulp.

6. The therapeutic composition according to claim 1, wherein said stem cells are cells of a pad-like soft tissue localized immediately at an apical side of an extracted immature tooth below a papilla.

7. The therapeutic composition according to claim 5, wherein a pad-like soft tissue of said soft tissue of a tooth is a cell formation from a disposition of an impacted and/or retained tooth in the development phase between occurrence of osseous alveolar fundus and completion of root formation.

8. The therapeutic composition according to claim 1, wherein said cell-free substance has a defined ratio of calcium to phosphate.

9. The therapeutic composition according to claim 1, wherein said cell-free substance has a defined ratio of calcium phosphate to collagen.

10. A method for producing a therapeutic composition comprising stem cells and/or precursor cells that are isolated, cultivated in vitro and stimulated for subsequent differentiation, wherein after differentiation, said cells are killed and/or at least partially removed for producing a cell-free substance, and wherein said cell-free substance is prepared for therapeutic use.

11. The method according to claim 10, wherein said cell-free substance is disintegrated, pulverized and/or freeze-dried.

12. The method according to claim 10, wherein said cell-free substance is prepared in form of a paste, an ointment or a suspension.

13. The method according to claim 10, wherein said stem cells and/or precursor cells are osteogenically and/or chondrogenically stimulated.

14. The method according to claim 13, wherein duration of the stimulation is varied to set certain properties of said cell-free substance.

15. The method according to claim 13, wherein a defined ratio of calcium to phosphate and/or of calcium phosphate to collagen is set by selecting a certain duration of stimulation.

16. The method according to claim 10, wherein said stem cells and/or precursor cells are stimulated in such manner that they form a bone or cartilage matrix.

17. The method according to claim 16, wherein intact or disintegrated bone or cartilage matrix is treated for disrupting cells with a hypotonic solution and/or in liquid nitrogen.

18. The method according to claim 17, wherein said disrupted cells are washed under shaking in saline solution and/or acid/base and/or treated with Triton-X100 and/or SDS.

19. The method according to claim 17, wherein said disrupted cells are treated with RNAse and DNAse.

20. The method according to claim 17, wherein said disrupted cells are washed with a buffer solution and/or dialyzed.

21. The method according to claim 10, wherein said stem cells are isolated from a soft tissue of a tooth, preferably follicle or pulp.

22. The method according to claim 10, wherein said stem cells are isolated from a pad-like soft tissue localized immediately at an apical side of an extracted immature tooth below a papilla.

23. The method according to claim 22, wherein said pad-like soft tissue is obtained from a disposition of an impacted and/or retained tooth in a developmental phase between occurrence of osseous alveolar fundus and completion of the root formation.

24. The method according to claim 22, wherein said pad-like soft tissue, after surgical removal of the tooth, is separated from the tooth along a macroscopically visible border between the pad-like soft tissue and the papilla.

25. A therapeutic composition produced according to the method of claim 10.

26. A cell-free bone matrix produced using the method of claim 10.

27. A cell-free cartilage matrix produced using the method of claim 10.

28. Tissue replacement therapy comprising administering to a patient in need thereof an amount of a cell-free substance effective to enable or support an effective tissue replacement therapy.

29. Tissue replacement therapy comprising administering to a patient in need thereof an amount of a cell-free cell extract and/or cell secretion effective to enable or support an effective tissue replacement therapy.

30. Tissue replacement therapy comprising administering to a patient in need thereof an amount of cell-free bone or cartilage matrix effective to enable or support an effective tissue replacement therapy.
